(19) United States
(12) Patent Application Publication
(10) Pub. No.: US 2019/0313906 A1
(43) Pub. Date: Oct. 17, 2019

(54) ONE-TOUCH DATA TRANSMISSION MODULE AND METHOD THEREOF

(71) Applicants: Jin-Ping Chen, Taichung City 404 (TW); Chin-Hung Chen, Taichung City 404 (TW)

(72) Inventor: Jin-Ping Chen, Taichung City (TW)

(21) Appl. No.: 16/383,346

(22) Filed: Apr. 12, 2019

(30) Foreign Application Priority Data
Apr. 13, 2018 (CN) 201810331035.1

(51) Int. Cl. A61B 5/00; G16H 80/00; H04L 12/28

(52) U.S. Cl. CPC A61B 5/0022; H04L 12/2854; G16H 80/00; A61B 5/7475

(57) ABSTRACT

A one-touch data transmission module includes a data receiving unit, for receiving a transmission datum sent by a physiological data analyzer; a control unit, electrically connected to the data receiving unit, for obtaining the transmission datum received by the data receiving unit; a memory unit, electrically connected to the control unit, for storing the transmission datum; a communication unit, electrically connected to the control unit, for sending the transmission datum to a designated place; a power unit, for supplying power to the data receiving unit, the memory unit and the communication unit; a trigger button, electrically connected to the control unit, for triggering the control unit to start the data receiving unit to receive the transmission datum, the received transmission datum being send by the communication unit. The data transmission module provides the functions of simple operation and real-time data transmission for an elderly person.

### FIELD OF THE INVENTION

The present invention relates to a method and a module for transmitting physiological data, and more particularly to a one-touch data transmission module and a method thereof for real-time monitoring through simple operation steps.

### BACKGROUND OF THE INVENTION

People pay more and more attention to health conditions nowadays. Therefore, most people have simple physiological data measurement instruments at home for detecting personal physiological data such as blood pressure, blood glucose or body fat and for analyzing the information to understand the health. However, in the past, the information was recorded by handwriting. As technology advances day by day, many biometric data measurement instruments can record measured data on a computer or smart phone via a wired or wireless means.

With the change of modern lifestyles, the technology used to take care of the elderly cannot be separated from related technologies such as mobile phones or tablets. For the elderly, it is quite difficult to transmit these physiological data through complicated steps. In particular, even if the mobile phone has been set up, the problem of setting change may occur due to inadvertent or unintentional operation. As a result, the hospital or the caregiver cannot monitor the physiological data of the patient or the elderly, which will affect the prevention and control of the patient or the elderly.

It is a hot topic in this field how to solve the above problems in order to accurately transmit these physiological data to a hospital or a designated user end, thereby allowing the hospital or the caregiver to immediately monitor, notify and prevent an accident.

Accordingly, the inventor of the present invention has devoted himself based on his many years of practical experiences to solve these problems.

### SUMMARY OF THE INVENTION

In view of the shortcomings of the conventional physiological data transmission, the primary object of the present invention is to provide a one-touch data transmission module and a method thereof, which can achieve the purpose of monitoring and notifying the physiological data of a patient or an elderly person, providing warning and precautionary effects.

According to one aspect of the present invention, a one-touch data transmission module is provided. The one-touch data transmission module comprises a data receiving unit, a control unit, a memory unit, a communication unit, a power unit, and a trigger button. The data receiving unit is configured to receive a transmission datum sent by a physiological data analyzer. The control unit is electrically connected to the data receiving unit for obtaining the transmission datum received by the data receiving unit. The memory unit is electrically connected to the control unit for storing the transmission datum. The communication unit is electrically connected to the control unit for wirelessly sending the transmission datum to a signal receiving apparatus. The power unit is configured to supply power to the data receiving unit, the memory unit and the communication unit. The trigger button is electrically connected to the control unit for triggering the control unit to start the data receiving unit to receive the transmission datum, and then the received transmission datum is sent by the communication unit.

In the aforesaid one-touch data transmission module, the memory unit may store and drive an operating program of the one-touch data transmission module, and the operating program can be executed by the control unit. When the power supply is interrupted, the data stored in the memory will not disappear. After re-powering, the data in the memory can be read.

In the aforesaid one-touch data transmission module, the power unit includes a battery module and is electrically connected to the control unit. The power unit is configured to measure the performance of the battery module and output battery performance information.

Preferably, the one-touch data transmission module further comprises a display unit. The display unit is electrically connected to the control unit and configured to display the transmission datum, the basic analysis result, the advanced analysis result, the transmission result or a power status of the battery module on the display unit.

In the aforesaid one-touch data transmission module, the communication unit may be a low-power wide-area network (LPWAN) technology to achieve the transmission of the transmission datum.

Preferably, the one-touch data transmission module further comprises an environment detecting unit.

The transmission datum is a physiological datum or an environmental datum. The physiological data may include a blood glucose level, a blood pressure value, a body fat, a body weight, a body temperature, a basic analysis result, or an advanced analysis result. The environmental datum may include related environmental values, such as temperature, pressure, humidity or air quality.

According to another aspect of the present invention, a one-touch data transmission method is provided. The one-touch data transmission method comprises a data search pairing step, a data receiving step, and a data sending step. The data search pairing step is to search for a physiological data analyzer through a data receiving unit and perform a signal connection after searching. The data receiving step is to receive a transmission datum sent by the physiological data analyzer and store the transmission datum (including temporary storage) in a memory unit through a control unit. The data sending step is that the obtained transmission datum is sent to a signal receiving apparatus through a communication unit to perform low-power wide-area communication; and the transmission datum is further sent to a user terminal apparatus or a cloud server through the signal receiving apparatus.

In the aforesaid data transmission method, the user terminal apparatus may be a mobile phone, a tablet or a computer.

The user terminal apparatus can provide real-time physiological information for a hospital or a caregiver, and can monitor the physiological data of the patient or the elderly at any time to provide real-time notification and precautionary warning.

In the aforesaid data transmission method, in the data receiving steps, a display unit is provided. The display unit is electrically connected to the control unit. The display unit is configured to display the transmission datum or a power status of a battery module on the display unit.

In the aforesaid data transmission method, the transmission datum is a physiological datum or an environmental datum. The physiological datum may include a blood glucose level, a blood pressure value, a body fat, a body weight, a body temperature, a basic analysis result, or an advanced analysis result. The environmental datum may include related environmental values, such as temperature, pressure, humidity or air quality.

Embodiments of the present invention will now be described, by way of example only, with reference to the accompanying drawings.

### BRIEF DESCRIPTION OF THE DRAWINGS

FIG. 1 is a block diagram of a one-touch data transmission module according to an embodiment of the present invention;

FIG. 2 is a flowchart of a data transmission method according to an embodiment of the present invention; and

FIG. 3 is a block diagram of a one-touch data transmission module according to another embodiment of the present invention.

### DESCRIPTION OF THE PREFERRED EMBODIMENTS

Advantages and features of the inventive concept and methods of accomplishing the same may be understood more readily by reference to the following detailed description of embodiments and the accompanying drawings. The inventive concept may, however, be embodied in many different forms and should not be construed as being limited to the embodiments set forth herein.

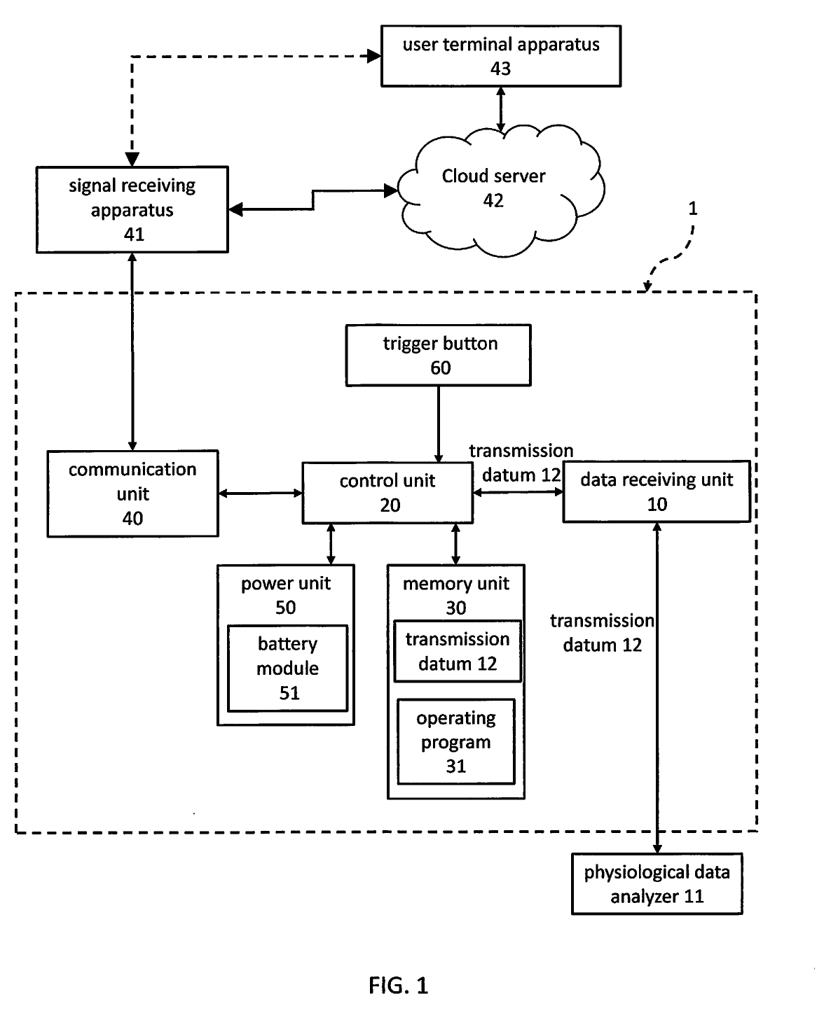

FIG. 1 is a schematic diagram of a one-touch data transmission module according to an embodiment of the present invention.

As shown in FIG. 1, a one-touch data transmission module 1 of the present invention comprises a data receiving unit 10, a control unit 20, a memory unit 30, a communication unit 40, a power unit 50, and a trigger button 60.

The control unit 20 is electrically connected to the data receiving unit 10, the memory unit 30, the communication unit 40, the power unit 50 and the trigger button 60. After the user presses the trigger button 60, the control unit 20 will start the corresponding operations as follows.

First, the data receiving unit 10 is configured to receive a transmission datum 12 sent by a physiological data analyzer 11. Wherein, the receiving mode includes a wireless or wired signal connection. For example, WiFi, Bluetooth, NFC, USB, Lightening or RS232 can be applied to the wired or wireless transmission described in this embodiment. The physiological data analyzer 11 may be a blood glucose meter, a body fat monitor, a weight scale, a thermometer, a blood pressure monitor, and any instrument that can be used for measuring or testing data to be used in this embodiment.

Then, the control unit 20 obtains the transmission datum 12 received by the data receiving unit 10 that is electrically to the control unit 20, and then performs the next processing step according to the content of the transmission datum 12. Furthermore, the content of the transmission datum 12 can be further automatically determined and directly transmitted to the corresponding unit according to the characteristics of the content. For example, if the user measures the blood glucose level, the blood glucose level will be transmitted to the corresponding medical patient database. If the user's weight change is detected, after the data of the weight measured is obtained, the control unit 20 transmits the data to the designated service unit after identification, without the troublesome procedure that the user needs to switch.

The memory unit 30 is configured to store the transmission datum 12 and drive an operating program 31 of the one-touch data transmission module 10. The operating program 31 may include related steps, such as the order of the operations and how to perform the search.

The communication unit 40 wirelessly sends the transmission datum 12 to a signal receiving apparatus 41. The signal receiving apparatus 41 may be a signal base station or a computer room of the relay station for transmitting the signal to a user terminal apparatus 43 or a cloud server 42.

The power unit 50 is configured to supply power to the data receiving unit 10, the memory unit 30 and the communication unit 40. The power unit 50 includes a battery module 51, which may be a rechargeable battery or a replaceable common battery. The power unit 50 is configured to measure the performance of the battery module 51 and output battery performance information.

After the trigger button 60 is pressed, the control unit 20 is triggered to start the data receiving unit 10 to receive the transmission datum or to start the operating program 31, and then the received transmission datum 12 is sent through the communication unit 40.

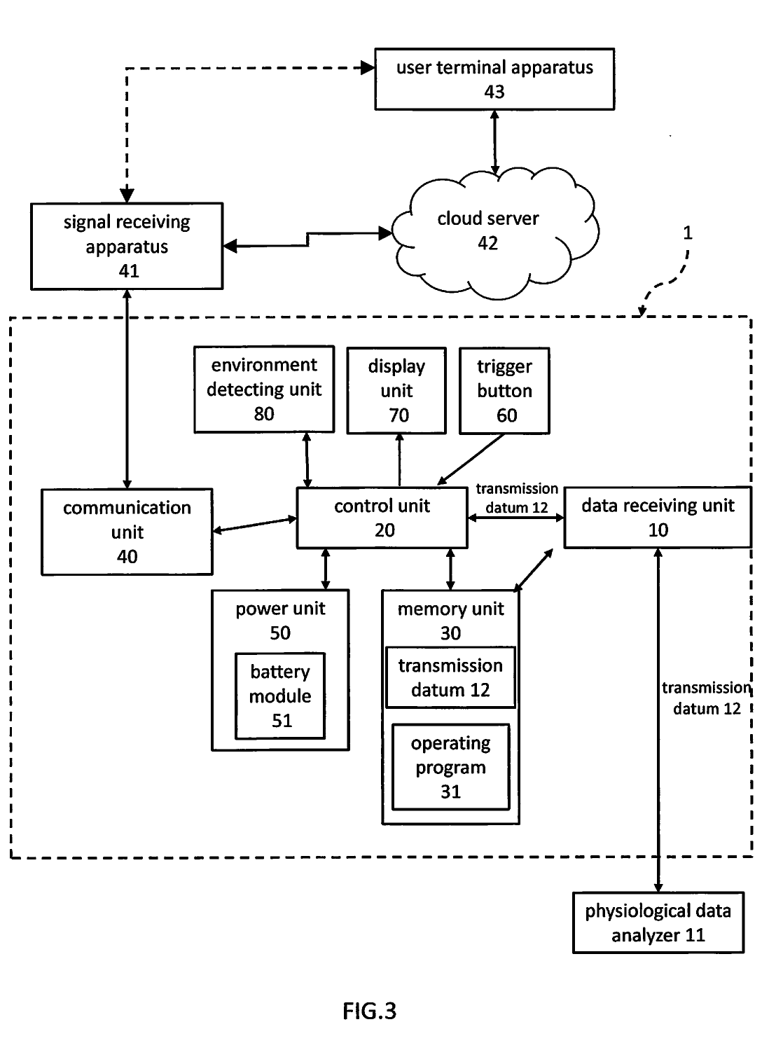

As shown in FIG. 3, the one-touch data transmission module 1 further comprises a display unit 70. The display unit 70 is electrically connected to the control unit 20 and configured to display the transmission datum 12, the basic analysis result, the advanced analysis result, the transmission result or a power status of the battery module 51 on the display unit 70.

The communication unit 40 may have a technical function including a low-power wide-area network or an existing communication network technology. The low-power wide-area network has the characteristics of low power demand and low bit rate, and can be used to distinguish the low-power wide-area network from a wireless wide-area network, thereby performing a wireless network capable of long-distance communication at a low bit rate, such as Sigfox, LoRa, NB-IoT and other wireless transmission technologies.

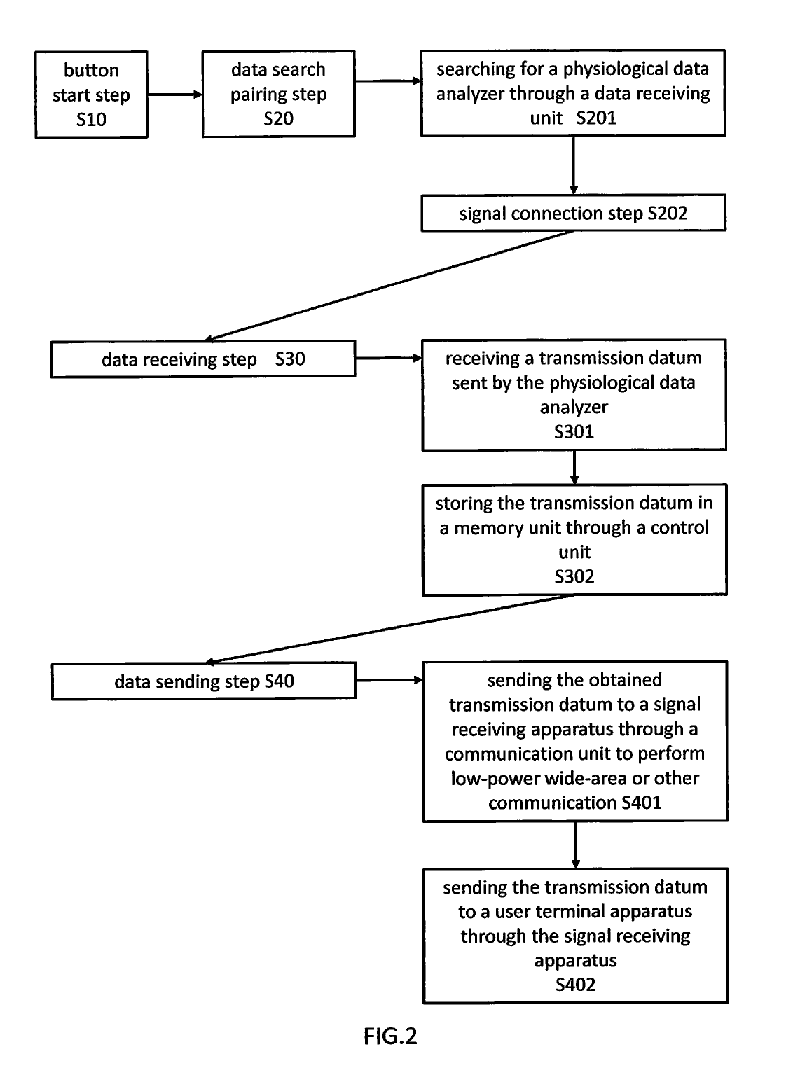

FIG. 2 is a flowchart of the data transmission method of the present invention.

In cooperation with the description of the components of the above one-touch data transmission module, a one-touch data transmission method of this embodiment comprises: a button start step S10, a data search pairing step S20, a data receiving step S30, and a data sending step S40.

First, the button start step S10 is that the user presses a push-button switch to start subsequent steps. The user does not need to press other settings, and only needs to press the preset button to start the related subsequent steps.

The data search pairing step S20 includes a step S201 and a step S202.

The step S201 is to search for a physiological data analyzer through a data receiving unit and perform signal connection and transmission after searching. Wherein, the search is performed through wireless or wired signals and further automatically determines the type of the connected physiological data analyzer when searching. After the search is completed, the signal connection step S202 is performed to complete the preparation of the relevant signal transmission.

The data receiving step S30 includes a step S301 and a step S302. Wherein, the step S301 is to receive a transmission datum sent by the physiological data analyzer 11. The step S302 is to store the transmission datum in a memory unit 30 through a control unit 20.

The data sending step S40 includes a step S401 and a step S402. The step S401 is that the obtained transmission datum is sent to a signal receiving apparatus 41 through a communication unit to perform low-power wide-area or other communication. The step S402 is that the transmission datum is further sent to a user terminal apparatus or a cloud server through the signal receiving apparatus.

Through the above method or module, the user can perform a one-touch operation, that is, the user only needs to press the trigger button 60 to start the method or module. The invention overcomes the problems of the prior art, and is convenient for the user to operate, and enhances the data transmission, so that the hospital or the family can confirm the condition in time and carry out early treatment and prevention and reduce an accident.

Although particular embodiments of the present invention have been described in detail for purposes of illustration, various modifications and enhancements may be made without departing from the spirit and scope of the present invention. Accordingly, the present invention is not to be limited except as by the appended claims.

### FIG. 1

(10) data receiving unit

(11) physiological data analyzer

(12) transmission datum

(20) control unit

(30) memory unit

(31) operating program

(40) communication unit

(41) signal receiving apparatus

(42) cloud server

(43) user terminal apparatus

(50) power unit

(51) battery module

(60) trigger button

### FIG. 2

button start step S10

data search pairing step S20

data receiving step S30

data sending step S40

searching for a physiological data analyzer through a data receiving unit S201

signal connection step S202

receiving a transmission datum sent by the physiological data analyzer S301

storing the transmission datum in a memory unit through a control unit S302

sending the obtained transmission datum to a signal receiving apparatus through a communication unit to perform low-power wide-area or other communication S401

sending the transmission datum to a user terminal apparatus through the signal receiving apparatus S402

### FIG. 3

(10) data receiving unit

(11) physiological data analyzer

(12) transmission datum

(20) control unit

(30) memory unit

(31) operating program

(40) communication unit

(41) signal receiving apparatus

(42) cloud server

(43) user terminal apparatus

(50) power unit

(51) battery module

(60) trigger button

(70) display unit

(80) environment detecting unit

What is claimed is:

1. A one-touch data transmission module, comprising:
a data receiving unit, for receiving a transmission datum sent by a physiological data analyzer;
a control unit, electrically connected to the data receiving unit, for obtaining the transmission datum received by the data receiving unit;
a memory unit, electrically connected to the control unit, for storing the transmission datum;
a communication unit, electrically connected to the control unit, for sending the transmission datum to a designated place;
a power unit, for supplying power to the data receiving unit, the memory unit and the communication unit;
a trigger button, electrically connected to the control unit, for triggering the control unit to start the data receiving unit to receive the transmission datum, the received transmission datum being further sent by the communication unit.

2. The one-touch data transmission module as claimed in claim 1, wherein the memory unit stores and drives an operating program of the one-touch data transmission module.

3. The one-touch data transmission module as claimed in claim 1, wherein the power unit includes a battery module and is electrically connected to the control unit, and the power unit is configured to measure performance of the battery module and output battery performance information.

4. The one-touch data transmission module as claimed in claim 1, further comprising a display unit electrically connected to the control unit.

5. The one-touch data transmission module as claimed in claim 1, further comprising an environment detecting unit electrically connected to the control unit for detecting environmental related values.

6. The one-touch data transmission module as claimed in claim 1, wherein the communication unit is a low-power wide-area network technology.

7. The one-touch data transmission module as claimed in claim 5, wherein the transmission datum is a physiological datum or an environmental datum.

8. A one-touch data transmission method, comprising:
a button start step, pressing a preset button to start subsequent steps;
a data search pairing step, searching for a physiological data analyzer through a data receiving unit and performing a signal connection;
a data receiving step, receiving a transmission datum sent by the physiological data analyzer and storing the transmission datum;
a data sending step, sending the obtained transmission datum to a signal receiving apparatus through a communication unit; the transmission datum being further sent to a user terminal apparatus or a cloud server through the signal receiving apparatus.

9. The one-touch data transmission method as claimed in claim 8, wherein in the data sending step, the transmission datum is sent by a low-power wide-area network technology.

10. The one-touch data transmission module as claimed in claim 8, wherein the transmission datum is a physiological datum or an environmental datum.
